(19) United States
(12) Patent Application Publication
(10) Pub. No.: US 2025/0295866 A1
(43) Pub. Date: Sep. 25, 2025

(54) A rotator for a medicament delivery device

(71) Applicant: SHL Medical AG, Zug (CH)

(72) Inventors: Justin Stewart, Deerfield Beach, FL (US); John Wiklund, Nacka Strand (SE)

(21) Appl. No.: 18/863,416

(22) PCT Filed: May 4, 2023
(86) PCT No.: PCT/EP2023/061739

§ 371 Date: Nov. 6, 2024

Related U.S. Application Data
(60) Provisional application No. 63/343,249, filed on May 18, 2022.

(30) Foreign Application Priority Data
Jun. 20, 2022 (EP) 22179818.4

(51) Int. Cl. A61M 5/32; A61M 5/20

(52) U.S. Cl. CPC A61M 5/3271; A61M 5/20; A61M 5/3245; A61M 2005/208; A61M 2005/3247; A61M 2005/3254; A61M 2205/0216

(57) ABSTRACT

The present disclosure provides a rotator for a medicament delivery device. The rotator includes a tubular body extending from a proximal end to a distal end relative to an axis and in a circumferential direction relative to the axis. The rotator also includes one or more ridges extending from a surface of the tubular body, the one or more ridges defining a track on the surface of the tubular body. The track extends from a distal end of the track to a proximal end of the track relative to the axis. The track includes one pathway at the distal end of the track and two pathways at the proximal end of the track. The two pathways at the proximal end of the track are separated by a ridge portion of the one or more ridges, and the ridge portion comprises a flexible arm.

### CROSS-REFERENCE TO RELATED APPLICATIONS

The present application is a U.S. National Phase Application pursuant to 35 U.S.C. § 371 of International Application No. PCT/EP2023/061739 filed May 4, 2023, which claims priority to (i) U.S. Provisional Application No. 63/43,249 filed May 18, 2022, and (ii) EP Patent Application No. 22168804.7 filed Jun. 20, 2022. The entire disclosure contents of these applications are herewith incorporated by reference into the present application.

### TECHNICAL FIELD

The invention concerns medicament delivery devices, and particularly rotators for medicament delivery devices.

### BACKGROUND

Medicament delivery devices such as those described in WO 2011/123024 have already been very commercially successful due to a combination of properties such as robustness, simplicity and usability. Nevertheless, the applicant has appreciated that there is still further scope for improvement of the mechanisms within medicament delivery devices such as those described in WO 2011/123024, to further optimise such mechanisms.

### SUMMARY

Reference should now be made to the appended claims.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

An aspect concerns a rotator for a medicament delivery device, the rotator comprising: a tubular body extending from a proximal end to a distal end relative to an axis and in a circumferential direction relative to the axis; and one or more ridges extending from a surface of the tubular body, the one or more ridges defining a track on the surface of the tubular body, the track extending from a distal end of the track to a proximal end of the track relative to the axis, the track comprising one pathway at the distal end of the track and two pathways at the proximal end of the track, wherein the two pathways at the proximal end of the track are separated by a ridge portion of the one or more ridges, and wherein the ridge portion comprises a flexible arm. Providing a flexible arm between the two pathways at the proximal end of the track can stop a protrusion of a medicament delivery member guard of a medicament delivery device comprising the rotator from returning back past the flexible arm after passing the flexible arm during activation of the medicament delivery device. This can make sure that, after medicament delivery is completed, the protrusion does not re-enter the pathway where the protrusion was previously situated. Another potential benefit of such a rotator is that it can create a point of no return, so that if the device is taken beyond a certain point in the activation process, it cannot return to the original position, and instead must continue to a final position in which the device is locked so that a medicament delivery member such as a needle cannot be left exposed. This can help ensure that a medicament delivery member such as a needle is not left exposed when the medicament delivery process is aborted.

Optionally, a proximal end of the flexible arm is attached to the rest of the rotator and a distal end of the flexible arm is free to flex relative to the rest of the rotator. Optionally, the flexible arm is arranged so that the flexible arm can flex in a circumferential direction relative to the axis. Optionally, the flexible arm is a plate with a first side and a second side, and each of the first side of the plate and the second side of the plate face in a circumferential direction relative to the axis. Optionally, the flexible arm is arranged so that the flexible arm can be flexed by a protrusion of a medicament delivery member guard when in use. Optionally, the flexible arm comprises a proximal portion that extends parallel to the axis and a distal portion that extends at an angle relative to the axis.

Another aspect concerns a medicament delivery device comprising any rotator as described above. Optionally, the medicament delivery device is an autoinjector. Optionally, the medicament delivery device comprises a medicament delivery device activation component. Optionally, the medicament delivery device activation component is a medicament delivery member guard. Optionally, the medicament delivery device activation component comprises a protrusion, and the protrusion is in the track defined by the one or more ridges of the rotator.

Optionally, the track on the surface of the tubular body is shaped so that a distal movement of the medicament delivery device activation component of the medicament delivery device relative to the rotator results in the protrusion moving from a first of the two pathways at the proximal end of the track to the pathway at the distal end of the track by flexing the flexible arm.

Optionally, the medicament delivery member guard is telescopically arranged inside a housing so that the medicament delivery member guard can move in the distal direction relative to the housing for activation of medicament delivery. Optionally, the medicament delivery member guard is rotationally restricted relative to the housing. Optionally, the rotator is arranged inside the housing so that the rotator is restricted from movement relative to the housing in the direction of the axis and is able to rotate relative to the housing when the medicament delivery member guard is moved in the distal direction.

Optionally, the flexible arm is arranged so that, during use of the medicament delivery device, the protrusion moves out of a first of the two pathways at the proximal end of the track and is restricted from moving back into the first of the two pathways at the proximal end of the track by the flexible arm.

The rotator would typically be manufactured as a single integral component. Optionally, the flexible arm could be a separate part and could be attached on to a rotator; this could allow existing rotators to be upgraded to include the flexible arm.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIG. 1 shows a perspective view of a rotator.

FIG. 2 shows a line diagram of an example track.

FIG. 3 shows a perspective view of a medicament delivery member guard that could be used with the rotator of FIG. 1.

FIG. 4 shows a side view of an autoinjector that could comprise the rotator of FIG. 1 and the medicament delivery member guard of FIG. 4.

FIG. 5 shows a close-up of part of the structure of the rotator of FIG. 1.

### DETAILED DESCRIPTION

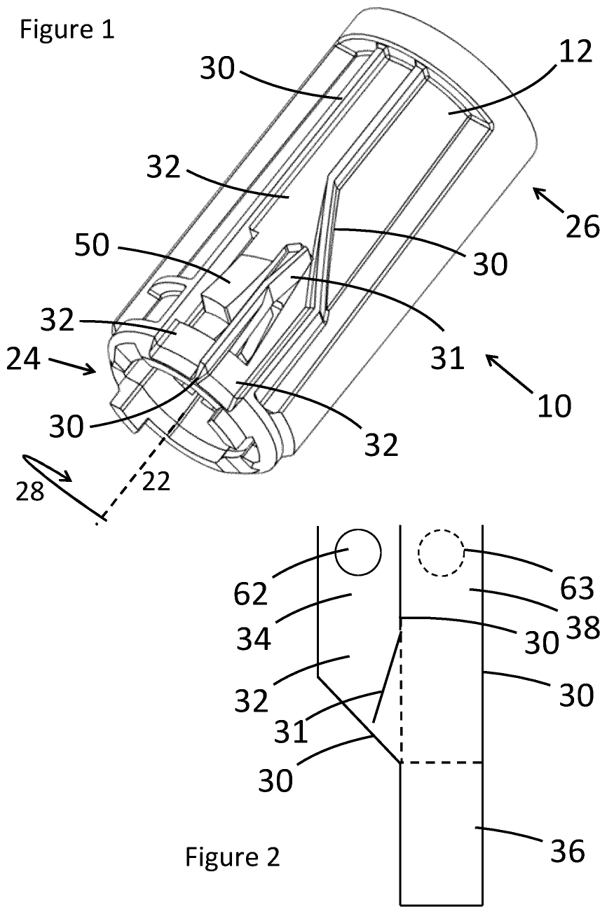

An example rotator 10 for a medicament delivery device is shown in FIG. 1. The rotator 10 comprises a tubular body 12 extending from a distal end 26 to a proximal end 24 relative to an axis 22 and in a circumferential direction 28 relative to the axis 22 and one or more ridges 30 extending from a surface of the tubular body 12. The one or more ridges 30 define a track 32 (labyrinth) on the surface of the tubular body 12. The track 32 extends from a distal end of the track to a proximal end of the track relative to the axis. The track 32 comprises one pathway 36 at the distal end of the track 32 and two pathways 34, 38 at the proximal end of the track 32. The two pathways 34, 38 at the proximal end of the track 32 are separated by a ridge portion of the one or more ridges 30. The ridge portion comprises a flexible arm 31. An optional tongue 50 is also provided, which can provide a medicament delivery member guard lock-out after use in a medicament delivery device comprising the rotator 10, by restricting distal movement of a medicament delivery member guard relative to the rotator 10.

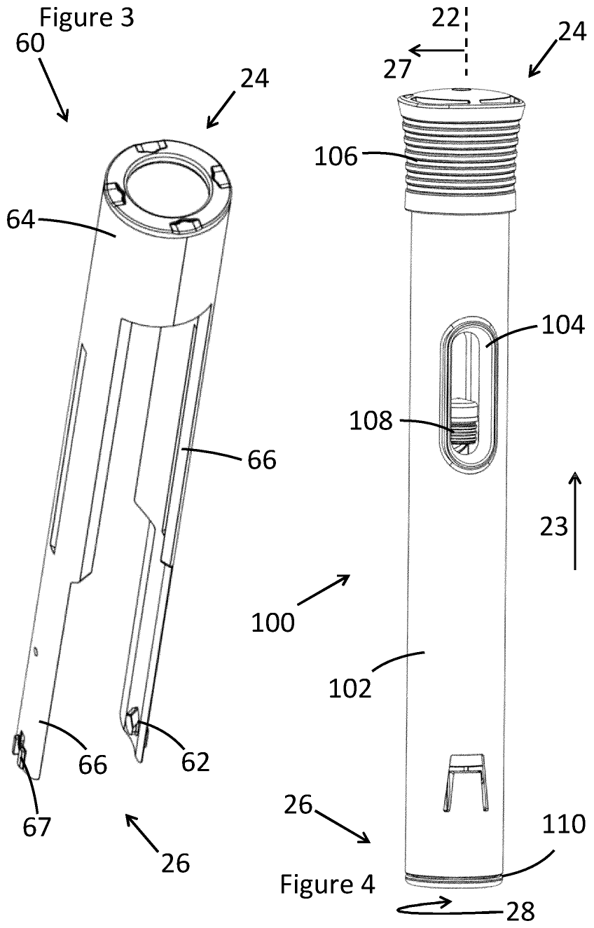

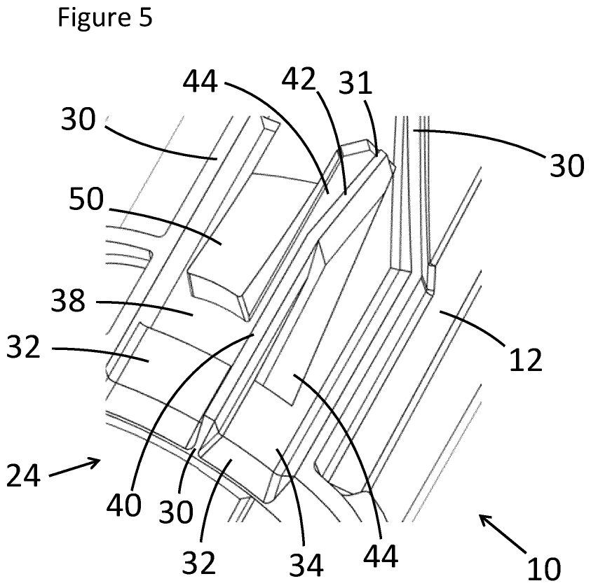

FIG. 2 shows a line diagram of an example track 32 similar to that in FIG. 1 to illustrate the structure of the track. The protrusion 62 of the medicament delivery member guard 60 (see FIG. 3) would initially be at the position shown in FIG. 2 in an assembled and unused medicament delivery device. When an injection is carried out using the medicament delivery device, the medicament delivery member guard moves in the distal direction relative to other parts of the device (relative to the rotator and relative to a housing, for example). In other words, relative to the medicament recipient, the medicament delivery member guard remains stationary with the proximal end of the medicament delivery member guard against the dose delivery site while the other parts of the device move in the proximal direction towards the dose delivery site. As the medicament delivery member guard moves relative to the rotator, the protrusion 62 also moves relative to the rotator and is arranged in the track so that it follows the track, firstly along the first pathway 34 and then along the second pathway 36. The medicament delivery member guard is typically rotationally fixed relative to other parts of the medicament delivery device (e.g. an outer housing, such as the housing 102 shown in FIG. 4, which will be used here as an example; in this example protrusions 67 on the arms 66 of the medicament delivery member guard 60 interact with corresponding grooves or ribs (not shown) of the housing to keep the medicament delivery member guard rotationally fixed relative to the housing, though other solutions could also be used for this rotational fixation, including other combinations of grooves, protrusions and/or ribs, and including rotational fixation relative to other components). During the initial movement of the protrusion in the first pathway, the medicament delivery member guard moves axially relative to the housing and the rotator remains stationary relative to the housing. As the protrusion transfers to the second pathway 36, the medicament delivery member guard continues to move axially relative to the housing, and the rotator also rotates (in the circumferential direction) relative to the housing and the medicament delivery member guard. The protrusion 62 also interacts with the flexible arm 31 at this stage, pushing the flexible arm 31 out of the way. Optionally, the release of the flexible arm by the protrusion 62 (resulting in the flexible arm suddenly relaxing back towards its unbiased position) could provide an indication that an injection is starting, for example by vibrating and/or by striking the adjacent part of the ridge 30. For example, this could be an indication either that the injection is about to start, that it is starting, or that it has just started, depending on the exact relative positions of the various parts of the medicament delivery device.

Roughly speaking, the protrusion is in the first pathway before injection, in the second pathway during injection and in the third pathway after injection, although the location of the protrusion during the precise transition points (from before to during to after injection) can vary somewhat depending on device design and use, so these transitions do not necessarily happen precisely when the protrusion travels from the first pathway to the second pathway and from the second pathway to third pathway—and indeed, injection completion normally occurs with the protrusion still in the second pathway. In more detail: immediately before injection, the protrusion is in the first pathway, and during the subsequent movement of the protrusion in the first pathway, the medicament delivery member guard moves axially relative to the housing and the rotator remains stationary relative to the housing. As the protrusion transfers to the second pathway 36, the medicament delivery member guard continues to move axially relative to the housing, and the rotator also rotates (in a circumferential direction) relative to the housing and the medicament delivery member guard. The point at which the injection would start would typically be with the protrusion in a distal part of the first pathway 34 or in the second pathway 36. During the actual injection, the medicament delivery member guard, the rotator and the housing would typically be stationary relative to one another. Once the injection is finished (which is typically indicated by an end click generated elsewhere within the medicament delivery device), the medicament delivery device is removed from the injection site, and as a result the medicament delivery member guard will move axially relative to the rotator and the housing in the opposite direction to the initial relative movement. As a result, the protrusion will move back along the second pathway and into the third pathway 38. The final position of the protrusion after injection would typically be at or near the position shown in a dashed line and indicated with the reference numeral 63 in FIG. 2. The third pathway is optional but can be beneficial, for example because the third pathway can allow the medicament delivery member guard to extend again after medicament delivery to cover the medicament delivery member, and because it can also provide a medicament delivery member guard lock out after use.

FIG. 3 shows an example of a medicament delivery member guard 60 that could be used with a rotator as described herein. The medicament delivery member guard 60 comprises a proximal portion 64 (in this example the proximal portion 64 is tubular and is cylindrical, though the shape could be varied, and could have a cross-section perpendicular to the axis 22 that is a different shape, rather than being round as in the depicted example) and two arms 66 that extend from the distal end of the proximal portion 64. Each arm comprises an inwardly extending protrusion 62.

The rotator described above is typically a standalone component in a medicament delivery device such as an autoinjector, an example of which is shown in FIG. 4. For reference, the axis 22, a radial direction 27 relative to the axis, a proximal direction 23 and a circumferential direction 28 are shown in FIG. 4. An example of a type of medicament delivery device the rotator could be used in is described in WO 2011/123024, which is herein incorporated by reference, particularly with reference to FIGS. 1 and 2 and the corresponding description. Briefly, a medicament delivery device incorporating the rotator would comprise a housing (such as an outer housing), a medicament delivery device activation component and the rotator; other components could be provided depending on the other functionality required for the medicament delivery device. The medicament delivery device activation component is a medicament delivery member guard in the depicted example. The rotator is able to move rotationally (in a circumferential direction) within the housing during use of the medicament delivery device. The medicament delivery member guard is telescopically arranged inside the housing (i.e. able to move in the direction of the axis within the housing, at least during use of the medicament delivery device). Typically, axial movement of the rotator relative to the housing would be restricted. Typically, rotational movement of the medicament delivery member guard relative to the housing would be restricted. The medicament delivery device also typically comprises a powerpack inside the housing (the powerpack comprising the rotator, a power source such as a spring or a battery, and a plunger rod), a medicament barrel inside the housing (the medicament barrel typically having a plunger inside it to hold a medicament in the medicament barrel), and a cap. The medicament barrel typically comprises a medicament container and a medicament delivery member such as a needle. Alternatively, a different medicament delivery member, such as a jet injector, could be used. The medicament delivery device may comprise a rear cap in addition to the housing; the rear cap would typically be part of the powerpack. In the example shown in FIG. 4, an autoinjector 100 comprises a housing 102 (comprising an optional window 104), an optional cap 106, a plunger 108 (visible through the window 104), and a rear cap 110.

FIG. 5 shows an expanded view of part of the rotator of FIG. 1. As can be seen, the flexible arm 31 comprises a proximal portion 40 and a distal portion 42. The proximal portion 40 extends parallel to the axis, and the distal portion 42 extends at an angle relative to the axis. The flexible arm 31 is in a cavity 44 in the surface of the track 32, thereby allowing the flexible arm 31 to be free to flex relative to the rest of the rotator, though the flexible arm could alternatively be arranged adjacent to the surface of the track, thereby removing the need for a cavity. The flexible arm in this example comprises two straight sections at an angle from one another, although this is optional and the flexible arm could be another shape, such as curved or a single straight section as shown in FIG. 2, for example.

In the example shown in FIG. 5, the distal portion 42 of the flexible arm 31 is angled towards the first pathway 34; this can be beneficial as it can make it harder for the protrusion 62 to accidentally re-enter the first pathway after leaving the first pathway during use of a medicament delivery device. Optionally, the distal portion 42 of the flexible arm 31 is angled relative to the axis by at least 5 degrees, or at between 5 and 60 degrees, or at between 10 and 45 degrees. In this example, the flexible arm is a plate, with each side of the plate facing in a circumferential direction, though other shapes, such as a rod shape, could alternatively be used. The flexible arm is shown as being attached to the rest of the rotator at the proximal end of the flexible arm, although the flexible arm could alternatively be attached to the rest of the rotator in a different way, such as at the distal end of the flexible arm.

In FIG. 1, the rotator is shown extending entirely around the axis in a circumferential direction, so 360 degrees around the axis, but the rotator could also extend only part of the way round the axis. Various other structural features of the rotator can be seen in the examples, but these features are not essential to the invention described herein. The rotator could be made as a single integral piece or as two or more pieces joined together. The rotator is envisioned as comprising two tracks (two labyrinths) opposite one another relative to the axis 22 (and two corresponding arms 66 on the medicament delivery member guard 60), but one, three or more tracks could be provided on the rotator, with the number of arms 66 of the medicament delivery member guard 60 being amended accordingly.

The ridge 30 can be one single ridge or alternatively two or more separate portions adjacent to one another or spaced apart from one another. Some or all of the ridge or ridges can have further functions, such as providing structural support for the rotator. The ridge in FIG. 1 could be considered to be two separate portions (one straight section between the two pathways and another larger section extending around the two pathways). The ridge in FIG. 1 is shown as a series of straight sections, but the specific shape of the ridge shown in FIG. 1 is not essential; for example, curved portions could be used instead. The track 32 is shown extending from near the distal end of the rotator to the proximal end of the rotator, but the track can also extend the entire length of the rotator or be spaced apart from the distal end of the rotator and/or the proximal end of the rotator. In general, the track described in this application can be considered as a volume of space in which the protrusion can move bounded by the ridge. The surface of the track (which is the surface of the rotator adjacent to the track) would typically also limit movement of the protrusion in practice, although this is not necessarily required for the invention to function. When assembled within a complete medicament delivery device, the track would typically also be physically bounded opposite the surface of the track, e.g. by a housing, although this is also not necessarily needed to keep the protrusion within the track. The pathway at the distal end of the track is typically aligned in the direction of the axis with only one of the two pathways at the proximal end of the track. Any particular ridge portion is typically optional, as limitations to the movement of the rotator and the movement of the medicament delivery member guard can also be provided by other features of a medicament delivery device, such as features of a housing of a medicament delivery device. The track 32 is shown on outside surface of the tubular body 12, but could be on the inside surface instead (with the protrusions 62 being outwardly protruding from the medicament delivery member guard 60 rather than inwardly protruding).

The medicament delivery member guard comprises a protrusion 62, which may take various shapes other than the shapes shown in the examples in the Figures. Optionally, the distal face of the protrusion is curved, and the proximal face of the protrusion is pointed, with two linear surfaces either side of the point (as shown in FIG. 3), though this can be varied; one alternative is a circular protrusion as shown in FIG. 2.

Optionally, the medicament delivery member guard is blocked (or at least limited) from moving in the proximal direction relative to the rotator in a completed medicament delivery device until some kind of block is removed; for example, this block could be provided by a feature of a cap or could be provided by a part of another medicament delivery device component such as an activation button. Once the block is removed, the medicament delivery member guard is able to move in the distal direction relative to the rotator.

The delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Various modifications to the embodiments described are possible and will occur to those skilled in the art without departing from the invention which is defined by the following claims.

1-14. (canceled)

15. A rotator for a medicament delivery device, the rotator comprising:
a tubular body extending from a proximal end to a distal end relative to an axis and in a circumferential direction relative to the axis; and
one or more ridges extending from a surface of the tubular body,
the one or more ridges defining a track on the surface of the tubular body, the track extending from a distal end of the track to a proximal end of the track relative to the axis,
the track comprising one pathway at the distal end of the track and two pathways at the proximal end of the track, wherein the two pathways at the proximal end of the track are separated by a ridge portion of the one or more ridges, and
wherein the ridge portion comprises a flexible arm.

16. The rotator of claim 15, wherein a proximal end of the flexible arm is attached to the rest of the rotator and a distal end of the flexible arm is free to flex relative to the rest of the rotator.

17. The rotator of claim 15, wherein the flexible arm is arranged so that the flexible arm can flex in a circumferential direction relative to the axis.

18. The rotator of claim 15, wherein the flexible arm is arranged so that the flexible arm can be flexed by a protrusion of a medicament delivery member guard when in use.

19. The rotator of claim 15, wherein the flexible arm comprises a proximal portion that extends parallel to the axis and a distal portion that extends at an angle relative to the axis.

20. A medicament delivery device comprising the rotator of claim 15.

21. The medicament delivery device of claim 20, further comprising a medicament delivery device activation component.

22. The medicament delivery device of claim 21, wherein the medicament delivery device activation component is a medicament delivery member guard.

23. The medicament delivery device of claim 21, wherein the medicament delivery device activation component comprises a protrusion, and wherein the protrusion is in the track defined by the one or more ridges of the rotator.

24. The medicament delivery device of claim 21, wherein the track on the surface of the tubular body is shaped so that a distal movement of the medicament delivery device activation component of the medicament delivery device relative to the rotator results in the protrusion moving from a first of the two pathways at the proximal end of the track to the pathway at the distal end of the track by flexing the flexible arm.

25. The medicament delivery device of claim 22, wherein the medicament delivery member guard is telescopically arranged inside a housing so that the medicament delivery member guard can move in the distal direction relative to the housing for activation of medicament delivery.

26. The medicament delivery device of claim 22, wherein the medicament delivery member guard is rotationally restricted relative to the housing.

27. The medicament delivery device of claim 22, wherein the rotator is arranged inside the housing so that the rotator is restricted from movement relative to the housing in the direction of the axis and is able to rotate relative to the housing when the medicament delivery member guard is moved in the distal direction.

28. The medicament delivery device of claim 23, wherein the flexible arm is arranged so that, during use of the medicament delivery device, the protrusion moves out of a first of the two pathways at the proximal end of the track and is restricted from moving back into the first of the two pathways at the proximal end of the track by the flexible arm.
